(19) United States
(12) Patent Application Publication
(10) Pub. No.: US 2024/0327484 A1
(43) Pub. Date: Oct. 3, 2024

(54) IL-12/IL-18 SUPERKINES

(71) Applicant: ImmunityBio, Inc., Culver City, CA (US)

(72) Inventors: Shiho Tanaka, Culver City, CA (US); Clifford Anders Olson, Culver City, CA (US); Wendy Higashide, Culver City, CA (US); Manju Saxena, Culver City, CA (US)

(21) Appl. No.: 18/607,825

(22) Filed: Mar. 18, 2024

Related U.S. Application Data
(60) Provisional application No. 63/493,443, filed on Mar. 31, 2023.

(51) Int. Cl. C07K 14/54; C07K 14/715; C12N 5/0783

(52) U.S. Cl. CPC C07K 14/5443; C07K 14/5434; C07K 14/7155; C12N 5/0646; C07K 2319/30; C12N 2501/2315; C12N 2501/39

(57) ABSTRACT

Provided herein are novel soluble-fusion protein complexes comprising interleukin 18 (IL-18) and interleukin 12 (IL-12) polypeptide domains. Also provided are compositions comprising the soluble-fusion protein complexes as well as methods of using the soluble-fusion protein complexes.

### CROSS-REFERENCE TO RELATED APPLICATIONS

This application claims the benefit of priority under 35 U.S.C. § 119 (e) to U.S. Provisional Patent Application No. 63/493,443, filed Mar. 31, 2023. The entire disclosure of U.S. Provisional Patent Application No. 63/493,443 is incorporated herein by reference.

### REFERENCE TO A SEQUENCE LISTING

This application contains a Sequence Listing submitted electronically as an ST.26 XML file. The XML file, named “PAT005342US002.xml”, has a size of 17,000 bytes, and was recorded on Mar. 18, 2024. The information contained in the XML file is incorporated herein by reference in its entirety pursuant to 37 CFR § 1.52 (e) (5).

### BACKGROUND

Therapeutic heteromer protein complexes, including the interleukin-15 (IL-15) superagonist N-803, have been found to modulate the immune response for treatment of various cancers. N-803 is a multimeric protein complex including an IL-15 mutant bound to an IL-15 receptor alpha (a), which is in turn attached to an immunoglobulin G1 (IgG1) crystallizable fragment (Fc). N-803 has been shown to display improved pharmacokinetic properties, longer persistence in lymphoid tissues and enhanced anti-tumor activity compared to native, non-complexed IL-15.

Continued evaluation of pharmacokinetic properties of therapeutic heteromer protein complexes such as N-803 as well as methods of making these complexes is critical for assessing clinical and manufacturing efficacy.

### SUMMARY

One embodiment relates to an isolated soluble-fusion protein complex comprising two soluble proteins, wherein the complex comprises: (a) a first soluble fusion protein comprising an: an interleukin-18 (IL-18) polypeptide domain; an IL-15 receptor alpha sushi-binding domain (IL-15RαSu); an immunoglobulin (Ig) crystallizable fragment (Fc) domain; and an IL-12 domain comprising p40 and p35 subunits; and (b) a second soluble protein comprising IL-15 (IL-15); wherein the IL-15-RαSu domain of the first soluble fusion protein binds to the IL-15 of the second soluble protein to form the soluble fusion protein complex; and wherein the immunoglobulin Fc domain of a first soluble protein complex is covalently linked to the Fc domain of a second soluble protein complex by a disulfide bond. In one aspect of this embodiment, the first soluble fusion protein comprises the amino acid sequence set forth in SEQ ID NO:5.

Another embodiment relates to an isolated soluble fusion protein complex comprising a soluble fusion protein, the soluble fusion protein comprising an interleukin-18 (IL-18) polypeptide domain, an immunoglobulin crystallizable fragment (Fc) domain, and an IL-12 polypeptide domain comprising p40 and p35 subunits; wherein the Fc domain of a first soluble protein is covalently linked to the Fc domain of a second soluble protein by a disulfide bond. In one aspect of this embodiment, the soluble fusion protein complex comprises the amino acid sequence set forth in SEQ ID NO:1

Another embodiment relates to a method of generating a memory-like cytokine enhanced natural killer (M-CENK) cell, comprising: obtaining a plurality of mononuclear cells, and contacting the plurality of mononuclear cells with a corticosteroid; incubating the plurality of mononuclear cells in the presence of the corticosteroid to enrich the mononuclear cells in NK cells; and inducing the enriched NK cells with either: an isolated soluble-fusion protein complex comprising two soluble proteins, wherein the complex comprises: a first soluble fusion protein comprising: an interleukin-18 (IL-18) polypeptide domain; an IL-15 receptor alpha sushi-binding domain (IL-15RαSu); an immunoglobulin (Ig) crystallizable fragment (Fc) domain; and an IL-12 polypeptide domain comprising p40 and p35 subunits; and a second soluble protein comprising IL-15 (IL-15); wherein the IL-15-RαSu domain of the first soluble fusion protein binds to the IL-15 of the second soluble protein to form the soluble fusion protein complex; and wherein the immunoglobulin Fc domain of a first soluble protein complex is covalently linked to the Fc domain of a second soluble protein complex by a disulfide bond, or an IL-15 or a derivative thereof and an isolated soluble fusion protein complex comprising a soluble fusion protein, the soluble fusion protein comprising an interleukin-18 (IL-18) polypeptide domain, an Ig crystallizable fragment (Fc) domain, and an IL-12 polypeptide domain comprising p40 and p35 subunits; wherein the Fc domain of a first soluble protein is covalently linked to the Fc domain of a second soluble protein by a disulfide bond, to generate the M-CENK cells.

In one aspect of the method, the corticosteroid is hydrocortisone.

In another aspect of the method, the IL-15 derivative is N-803.

In one aspect of any of the embodiments, the IL-15 polypeptide domain is an IL-15 variant comprising an asparagine-to-aspartate mutation at amino acid position 72 (IL-15N72D).

In one aspect of any of the embodiments, the IL-18 domain is linked to the IL-15RαSu domain by a flexible polypeptide linker.

In one aspect of any of the embodiments, the IL-18 polypeptide domain is linked by a flexible polypeptide linker to the Ig Fc domain.

In one aspect of any of the embodiments, the p40 and p35 subunits of the IL-12 domain are linked by a flexible polypeptide linker.

In one aspect of any of the embodiments, the IL-12 p40 subunit is linked by a flexible polypeptide linker to the Ig Fc domain.

### BRIEF DESCRIPTION OF THE DRAWINGS

FIGS. 1A-1E are schematics of the known IL-12/15/18 superkine (SK) (ALTOR BIOSCIENCE®) (IL-12/15/18 SK (Altor); FIG. 1A), the novel IL-12/15/18 SK (new) (FIG. 1B), the novel IL-18/15/12 SK (FIG. 1C), the novel IL-18/12 SK (FIG. 1D), and the novel IL-12/18 SK (FIG. 1E), soluble fusion protein complexes disclosed herein.

FIG. 1F is a graph showing the results of an IL-18 reported cell line activity assay using HEK-BLUE™ IL-18 cells (INVIVOGEN®) with the SK protein constructs shown in FIGS. 1A-1E.

FIGS. 2A-2C show HPLC chromatograms for the IL-12/15/18 (FIG. 2A), IL-18/15/12 (FIG. 2B) and IL18/12 (FIG. 2C) superkine protein complexes.

FIGS. 3A-3B show the results of an IL-18 reporter assay wherein IL-18/12 superkine design exhibited better IL-18 activity compared to the novel IL-18/15/12 superkine as well as a known IL-12/15/18 superkine protein complex known as HCW9201 (see FIG. 3B for schematic of HCW9201). HCW9201 schematic and EC50 value was taken from Becker-Hapak, M. K., Cancer Immunol Res, 9 (9). 2020.

FIGS. 4A and 4B are graphs showing the percentage of IFN-gamma+CD56+Cells with either the IL-18/12 superkine protein complex plus N-803 (FIG. 4A) or the IL-18/15/12 superkine protein complex (FIG. 4B). As seen in the graphs IL-18/12 superkine protein complex plus N-803 exhibited better M-CENK activation than IL-18/15/12 superkine protein complex.

FIG. 5 shows a graph of a pSTAT-5 FLOW assay with the novel IL-18/15/12 superkine protein complex disclosed herein compared to a known IL-12/15/18 protein complex (i.e. HCW9201-see FIG. 3B). The novel IL-18/15/12 superkine protein complex has a higher IL-15 activity than the known IL-12/15/18 protein complex (EC50 value for HCW9201 was taken from Becker-Hapak, M. K., Cancer Immunol Res, 9 (9). 2020. For the pSTAT-5-FLOW assay: IL2 aNK cells were stimulated with 8-concentrations of each construct for 20 minutes. Cells were fixed/permeabilized and stained by PE-anti-pSTAT5 for FLOW read.

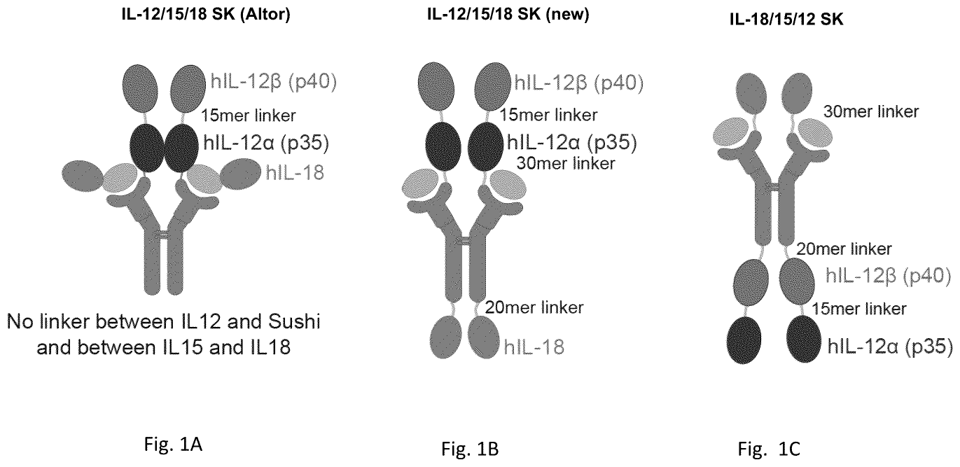

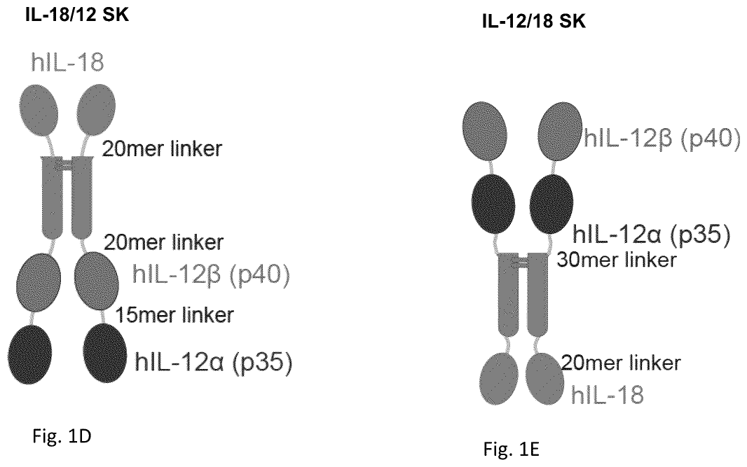

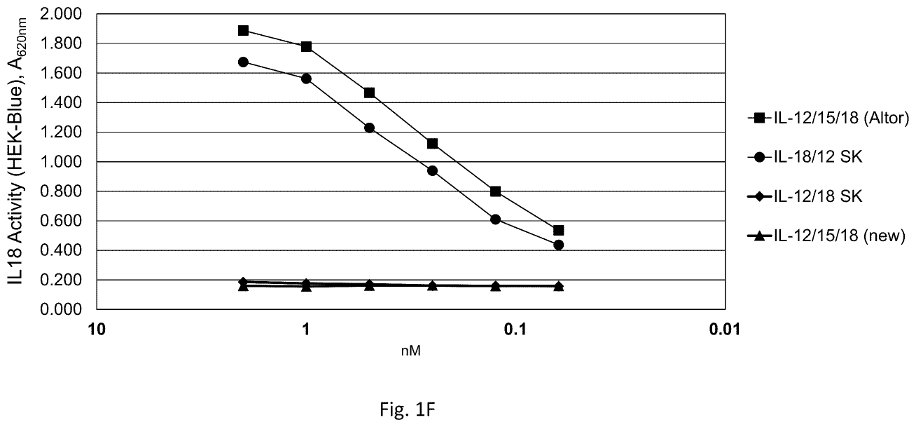

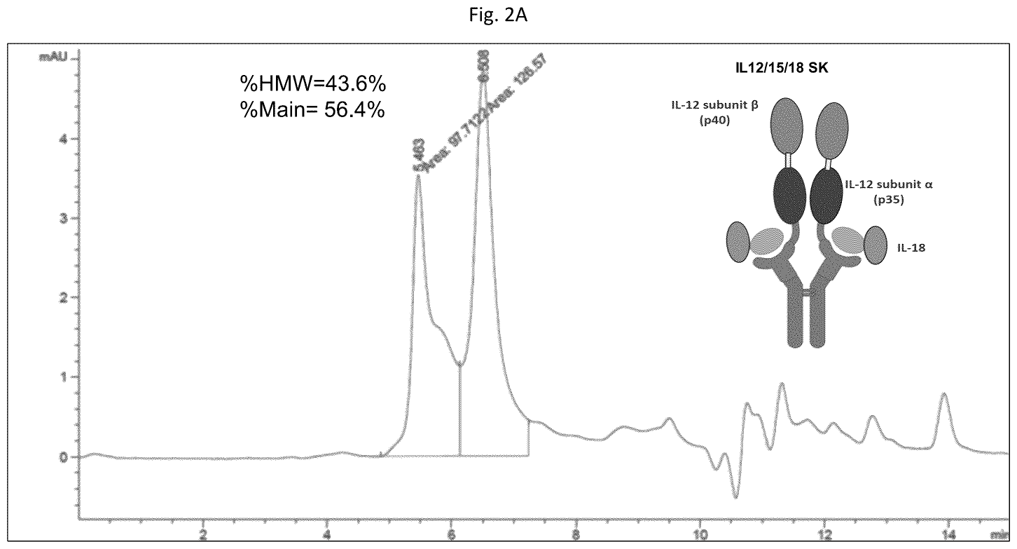

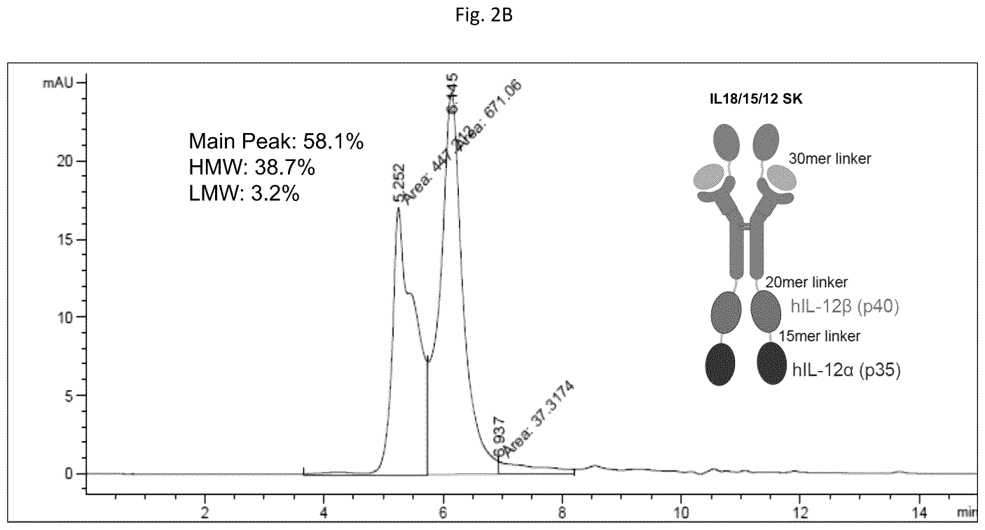

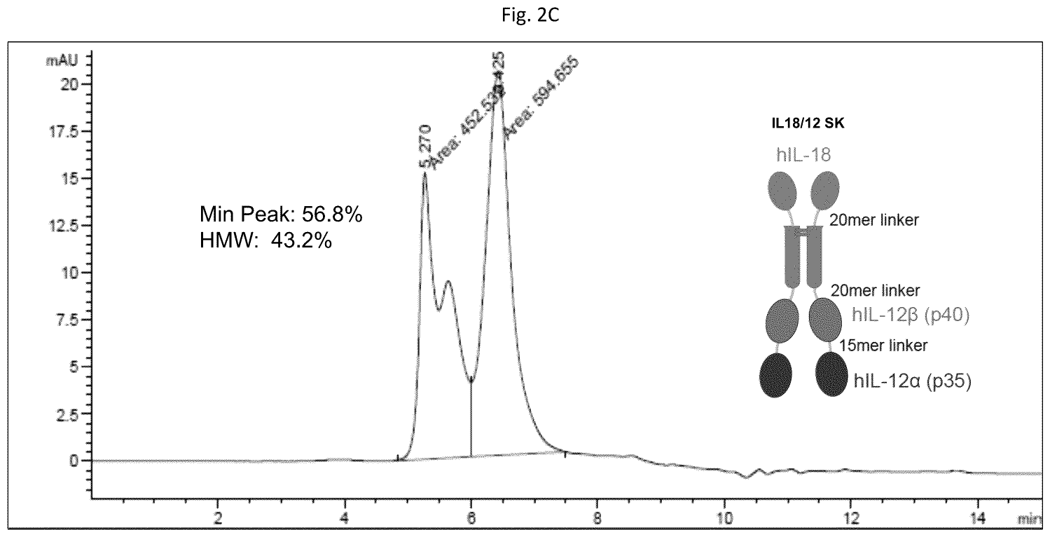

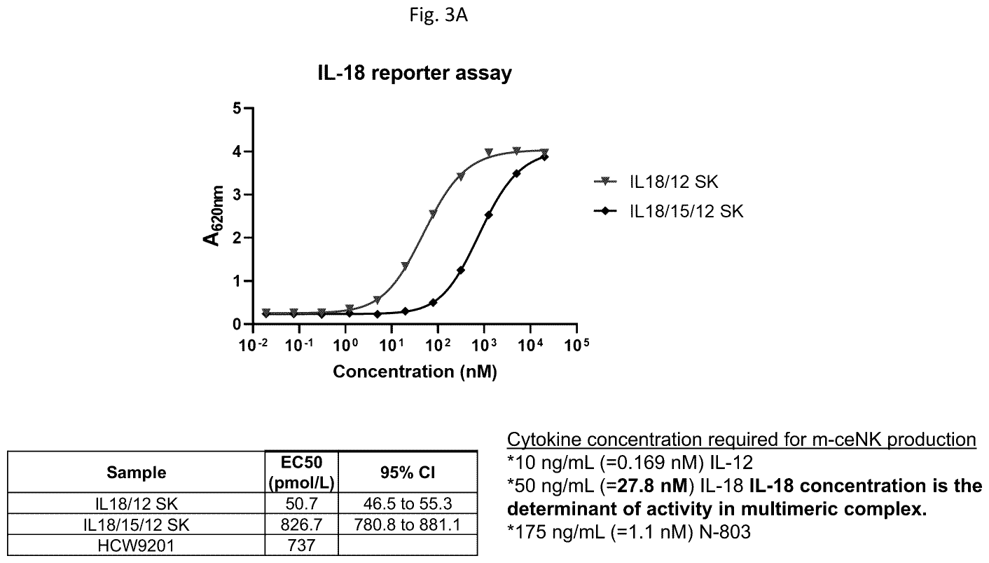

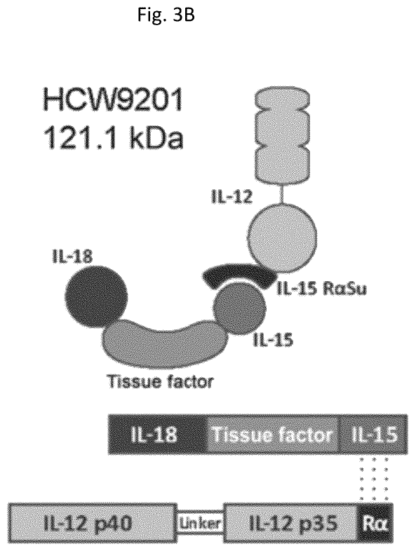

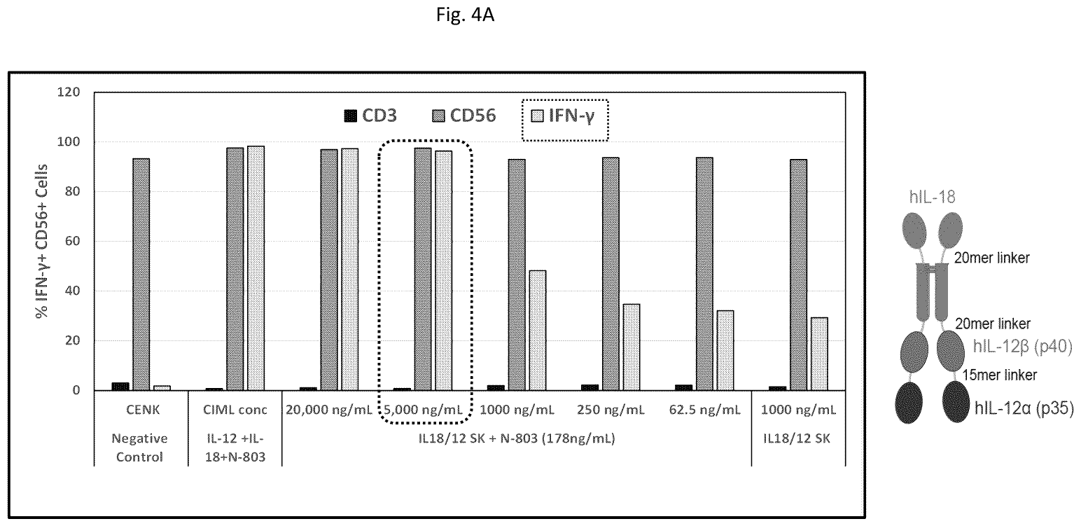

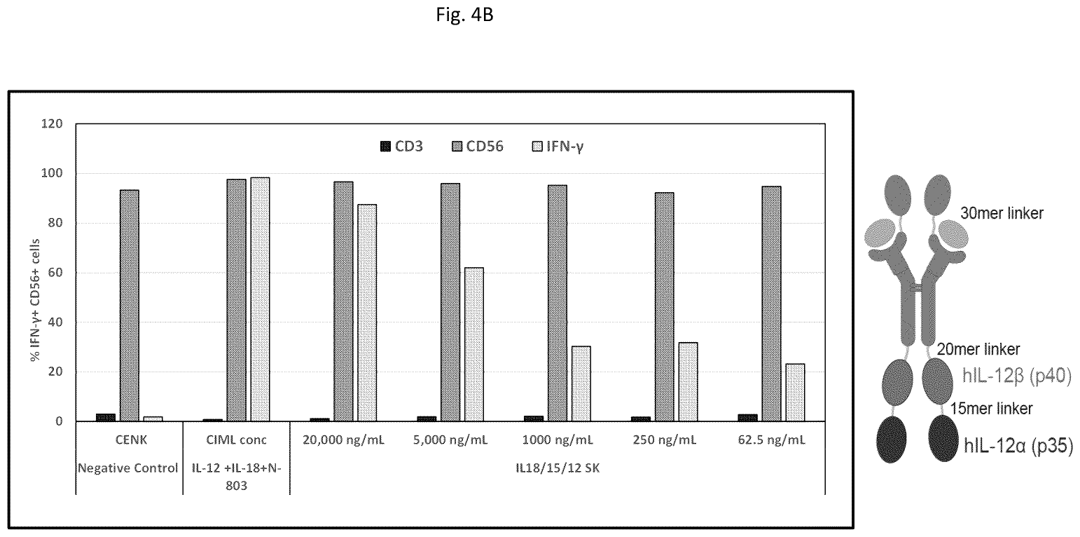

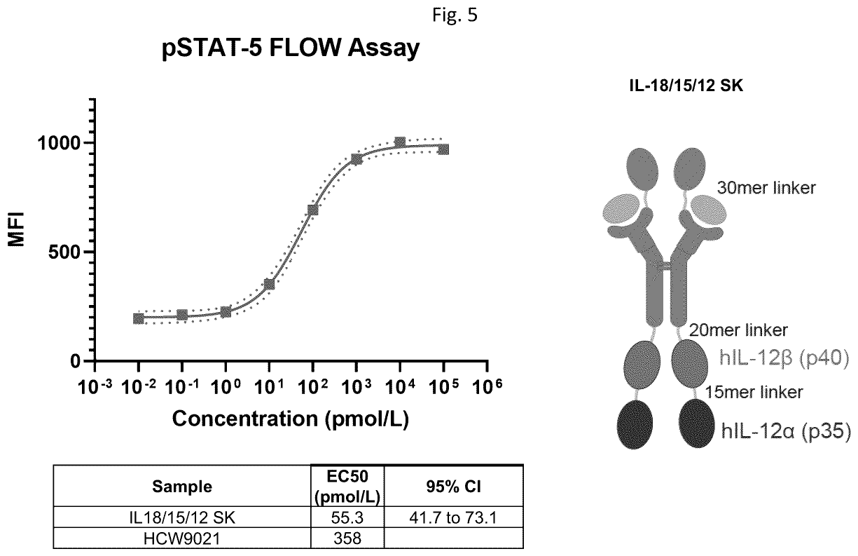

### DETAILED DESCRIPTION

After reading this description it will become apparent to one skilled in the art how to implement the present disclosure in various alternative embodiments and alternative applications. However, all the various embodiments of the present invention will not be described herein. It will be understood that the embodiments presented here are presented by way of an example only, and not limitation. As such, this detailed description of various alternative embodiments should not be construed to limit the scope or breadth of the present disclosure as set forth herein.

Before the present technology is disclosed and described, it is to be understood that the aspects described below are not limited to specific compositions, methods of preparing such compositions, or uses thereof as such may, of course, vary. It is also to be understood that the terminology used herein is for the purpose of describing particular aspects only and is not intended to be limiting.

The detailed description divided into various sections only for the reader's convenience and disclosure found in any section may be combined with that in another section. Titles or subtitles may be used in the specification for the convenience of a reader, which are not intended to influence the scope of the present disclosure.

### Definitions

Unless defined otherwise, all technical and scientific terms used herein have the same meaning as commonly understood by one of ordinary skill in the art to which this disclosure belongs. In this specification and in the claims that follow, reference will be made to a number of terms that shall be defined to have the following meanings:

The terminology used herein is for the purpose of describing particular embodiments only and is not intended to be limiting. As used herein, the singular forms “a”, “an” and “the” are intended to include the plural forms as well, unless the context clearly indicates otherwise.

“Optional” or “optionally” means that the subsequently described event or circumstance can or cannot occur, and that the description includes instances where the event or circumstance occurs and instances where it does not.

The term “about” when used before a numerical designation, e.g., temperature, time, amount, concentration, and such other, including a range, indicates approximations which may vary by (+) or (−) 10%, 5%, 1%, or any subrange or subvalue there between. Preferably, the term “about” when used with regard to an amount means that the amount may vary by +/−10%.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this disclosure.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. Typically, the antigen-binding region of an antibody plays a significant role in determining the specificity and affinity of binding. In some embodiments, antibodies or fragments of antibodies may be derived from different organisms, including humans, mice, rats, hamsters, camels, etc. Antibodies of the invention may include antibodies that have been modified or mutated at one or more amino acid positions to improve or modulate a desired function of the antibody (e.g. glycosylation, expression, antigen recognition, effector functions, antigen binding, specificity, etc.).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively.

As used herein, the terms “Fc domain” or “fragment crystallizable domain” are used in accordance with their plain and ordinary meanings and refer to any of the recombinant or naturally-occurring forms of the “base” or tail-end region (C-terminal) of an antibody. The Fc domain is typically composed of two heavy chains that contribute two or three constant domains depending on the class of the antibody. The Fc region is comprised of two heavy chain constant Ig domains in the antibodies IgG, IgA, and IgD, and of three heavy chain constant Ig domains in the antibodies IgE and IgM.

The term “Fc” refers to a non-antigen-binding fragment of an antibody. Such an “Fc” can be in monomeric or multimeric form. The original immunoglobulin source of the native Fc is preferably of human origin and may be any of the immunoglobulins. In embodiments, the Fc is an IgG1 or IgG2 Fc. Native Fc's are made up of monomeric polypeptides that may be linked into dimeric or multimeric forms by covalent (i.e., disulfide bonds) and non-covalent association. The number of intermolecular disulfide bonds between monomeric subunits of native Fc molecules ranges from 1 to 4 depending on class (e.g., IgG, IgA, IgE) or subclass (e.g., IgG1, IgG2, IgG3, IgA1, IgGA2). One example of a native Fc is a disulfide-bonded dimer resulting from papain digestion of an IgG (see Ellison et al. (1982), Nucleic Acids Res. 10:4071-9). The term “Fc” as used herein is generic to the monomeric, dimeric, and multimeric forms.

In embodiments, the term “Fc” refers to a molecule or sequence that is modified from a native Fc, but still comprises a binding site for a receptor. As with modified Fc and native Fc's, the term “Fc domain” includes molecules in monomeric or multimeric form, whether digested from whole antibody or produced by recombinant gene expression or by other means.

In embodiments, the Fc (or Fc domain) is attached, either directly or indirectly, to an IL-15Rα or an IL-15 domain. In embodiments, the Fc (or the Fc domain) is covalently and/or genetically fused with an IL-15Rα or an IL-15 domain. In still a further embodiment, the Fc (or the Fc domain) from one soluble fusion protein complex disclosed herein is covalently linked by a disulfide bond to the Fc (or Fc domain) of a second soluble protein complex comprising IL-15. In one aspect, the soluble fusion protein complex comprises a first soluble protein comprising an IL-18 polypeptide domain, an IL15RαSu domain, an Fc domain and an IL-12 domain comprising p40 and p35 subunits, and a second soluble protein comprising IL-15. In yet another embodiment, the soluble fusion protein complex comprises a soluble fusion protein comprising an IL-18 polypeptide domain, an Fc domain, and an IL-12 polypeptide domain comprising p40 and p35 subunits wherein the Fc domain of a first soluble protein is covalently linked to the Fc domain of a second soluble protein by a disulfide bond.

Antibodies exist, for example, as intact immunoglobulins or as a number of well-characterized fragments produced by digestion with various peptidases. Thus, for example, pepsin digests an antibody below the disulfide linkages in the hinge region to produce F(ab)′2, a dimer of Fab which itself is a light chain joined to VH-CH1 by a disulfide bond. The F(ab)′2 may be reduced under mild conditions to break the disulfide linkage in the hinge region, thereby converting the F(ab)′2 dimer into an Fab′ monomer. The Fab′ monomer is essentially the antigen binding portion with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

A single-chain variable fragment (scFv) is typically a fusion protein of the variable regions of the heavy (VH) and light chains (VL) of immunoglobulins, connected with a short linker peptide of 10 to about 25 amino acids. The linker may usually be rich in glycine for flexibility, as well as serine or threonine for solubility. The linker can either connect the N-terminus of the VH with the C-terminus of the VL, or vice versa.

The epitope of a mAb is the region of its antigen to which the mAb binds. Two antibodies bind to the same or overlapping epitope if each competitively inhibits (blocks) binding of the other to the antigen. That is, a 1×, 5×, 10×, 20× or 100× excess of one antibody inhibits binding of the other by at least 30% but preferably 50%, 75%, 90% or even 99% as measured in a competitive binding assay (see, e.g., Junghans et al., Cancer Res. 50:1495, 1990). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

The phrase “specifically (or selectively) binds” to an antibody or “specifically (or selectively) immunoreactive with,” when referring to a protein or peptide, refers to a binding reaction that is determinative of the presence of the protein, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “protein complex” or “complex” as used herein refers to two or more polypeptides that associate simultaneously. The complexes may be constructed through binding between proteins and/or binding between receptors and ligands. The proteins may be associated through non-covalent protein-protein interactions, though certain polypeptides in the complex may also be covalently linked directly or indirectly through, for example, a chemical linker, a bond or another protein. For example, the heterotetrameric IL-15/IL-15RαSu complex includes two IL-15 domains non-covalently bound to two IL-15RαSu domains, wherein the two IL-15RαSu domains are attached covalently by a disulfide bond.

A “detectable means” or “detectable moiety” is a composition, substance, element, or compound; or moiety thereof; detectable by appropriate means such as spectroscopic, photochemical, biochemical, immunochemical, chemical, magnetic resonance imaging, or other physical means.

For example, a detectable means includes ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra, ²²⁵Ac, Cr, V, Mn, Fe, Co, Ni, Cu, La, Ce, Pr, Nd, Pm, Sm, Eu, Gd, Tb, Dy, Ho, Er, Tm, Yb, Lu, ³²P, fluorophore (e.g. fluorescent dyes), electron-dense reagents, enzymes (e.g., as commonly used in an ELISA), biotin, digoxigenin, paramagnetic molecules, paramagnetic nanoparticles, ultrasmall superparamagnetic iron oxide (“USPIO”) nanoparticles, USPIO nanoparticle aggregates, superparamagnetic iron oxide (“SPIO”) nanoparticles, SPIO nanoparticle aggregates, monochrystalline iron oxide nanoparticles, monochrystalline iron oxide, nanoparticle contrast agents, liposomes or other delivery vehicles containing Gadolinium chelate (“Gd-chelate”) molecules, Gadolinium, radioisotopes, radionuclides (e.g. carbon-11, nitrogen-13, oxygen-15, fluorine-18, rubidium-82), fluorodeoxyglucose (e.g. fluorine-18 labeled), any gamma ray emitting radionuclides, positron-emitting radionuclide, radiolabeled glucose, radiolabeled water, radiolabeled ammonia, biocolloids, microbubbles (e.g. including microbubble shells including albumin, galactose, lipid, and/or polymers; microbubble gas core including air, heavy gas(es), perfluorcarbon, nitrogen, octafluoropropane, perflexane lipid microsphere, perflutren, etc.), iodinated contrast agents (e.g. iohexol, iodixanol, ioversol, iopamidol, ioxilan, iopromide, diatrizoate, metrizoate, ioxaglate), barium sulfate, thorium dioxide, gold, gold nanoparticles, gold nanoparticle aggregates, fluorophores, two-photon fluorophores, or haptens and proteins or other entities which can be made detectable, e.g., by incorporating a radiolabel into a peptide or antibody specifically reactive with a target peptide. A detectable means may be a monovalent detectable agent or a detectable agent capable of forming a bond with another composition.

As used herein, the term “conjugate” refers to the association between atoms or molecules. The association can be direct or indirect. For example, a conjugate between an antigen binding domain and a peptide compound can be direct, e.g., by covalent bond (e.g., a disulfide bond), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, conjugates are formed using conjugate chemistry including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated, however, the resulting reaction product can be produced directly from a reaction between the added reagents or from an intermediate from one or more of the added reagents that can be produced in the reaction mixture.

The term “contacting” may include allowing two species to react, interact, or physically touch, wherein the two species may be an antibody and a fusion protein, biological sample, etc. as described herein.

A “control” sample or value refers to a sample that serves as a reference, usually a known reference, for comparison to a test sample. For example, a test sample can be taken from a test condition (e.g., in the presence of a test compound), and compared to samples from known conditions (e.g., in the absence of the test compound (negative control), or in the presence of a known compound (positive control)). A control can also represent an average value gathered from a number of tests or results. One of skill in the art will recognize that controls can be designed for assessment of any number of parameters. For example, a control can be devised to determine a background level of signal (negative control) or an expected level of signal (e.g., a standard curve or positive control). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

“Biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The term “polymeric” refers to a molecule including repeating subunits (e.g., polymerized monomers). For example, polymeric molecules may be based upon polypropylene (PP), polystyrene (PS), polyethylene glycol (PEG), poly [amino (1-oxo-1,6-hexanediyl)], poly(oxy-1,2-ethanediyloxycarbonyl-1,4-phenylenecarbonyl), tetraethylene glycol (TEG), polyvinylpyrrolidone (PVP), poly(xylene), or poly(p-xylylene). See, for example, “Chemistry of Protein Conjugation and Cross-Linking” Shan S. Wong CRC Press, Boca Raton, Fla., USA, 1993; “BioConjugate Techniques” Greg T. Hermanson Academic Press, San Diego, Calif., USA, 1996; “Catalog of Polyethylene Glycol and Derivatives for Advanced PEGylation, 2004” Nektar Therapeutics Inc, Huntsville, Ala., USA, which are incorporated by reference in their entirety for all purposes.

An “interleukin-15 protein” or “IL-15” as referred to herein includes any of the recombinant or naturally-occurring forms of the interleukin-15 (IL-15) protein or variants or homologs thereof that maintain IL-15 protein activity (e.g. within at least 50%, 80%, 90%, 95%, 96%, 97%, 98%, 99% or 100% activity compared to IL-15 protein). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15 protein. In embodiments, the IL-15 protein is substantially identical to the protein identified by the UniProt reference number P40933 or a variant or homolog having substantial identity thereto.

An “interleukin-15 receptor subunit alpha protein” or “IL-15Rα” as referred to herein includes any of the recombinant or naturally-occurring forms of the interleukin-15 receptor subunit alpha (IL-15Rα) protein or variants or homologs thereof that maintain IL-15Rα protein activity (e.g. within at least 50%, 80%, 90%, 95%, 96%, 97%, 98%, 99% or 100% activity compared to IL-15Rα protein). In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring IL-15Rα protein. In embodiments, the IL-15Rα protein is substantially identical to the protein identified by the UniProt reference number Q13261 or a variant or homolog having substantial identity thereto.

As used herein, “domain” refers to a conserved portion of a protein that functions and exists independently of the rest of the protein sequence. A domain may form a stable, three-dimensional structure that exists as a functional unit independent of the remaining protein. For example, the IL-15RαSu domain is the portion of IL-15Rα that retains the IL-15 binding activity.

As used herein, “IL-15 domain” refers to a polypeptide comprising at least a portion of a sequence of the IL-15 protein. In embodiments, the IL-15 domain comprises at least a portion of the sequence of the IL-15 protein and includes one or more amino acid substitutions or deletions within the amino acid sequence of the IL-15 protein. In embodiments, the IL-15 domain is an IL-15 variant that comprises a different a different amino acid sequence compared to the IL-15 protein. In embodiments, the IL-15 domain binds the IL-15Rα protein or a fragment thereof. In embodiments, the IL-IS domain is bound to the IL-15Rα protein or a fragment thereof. In embodiments, the sequence of the IL-15 domain has at least one amino acid change, e.g. substitution or deletion, compared to the IL-15 protein. In embodiments, the amino acid substitutions/deletions are in the portions of IL-15 that interact with IL-15Rβ and/or γC. In embodiments, the amino acid substitutions/deletions do not affect binding to the IL-15Rα polypeptide or the ability to produce the IL-15 domain. In embodiments, amino acid substitutions can be conservative or non-conservative changes and insertions of additional amino acids compared to the IL-15 protein. In embodiments, the IL-15 domain comprises one or more than one amino acid substitutions/deletions at position 6, 8, 10, 61, 65, 72, 92, 101, 104, 105, 108, 109, 111, or 112 of the IL-15 protein sequence. In embodiments, the IL-15 domain comprises an N72D substitution of the IL-15 protein sequence (IL15N72D. SEQ ID NO:3 (amino acid sequence) and SEQ ID NO:4 (nucleic acid sequence).

The term “sushi domain” as used herein refers to a common motif in proteins comprising a beta-sandwich arrangement. Sushi domains are common in protein-protein interactions, and typically include four cysteines forming two disulfide bonds in a 1-3 and 2-4 pattern. For example, the region of IL-15Rα that binds IL-15 includes a sushi domain.

In embodiments, the IL-15Rα sushi domain includes the amino acid sequence comprising the sequence of SEQ ID NO:7. In embodiments, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to the sequence of SEQ ID NO:7. In embodiments, the IL-15Rα sushi domain associates with the IL-15 protein. In embodiments, the IL-15Rα sushi domain associates with the IL-15 domain.

### Soluble Fusion Proteins, Compositions and Methods

In one aspect, an isolated soluble-fusion protein complex is provided comprising two soluble proteins, wherein the complex comprises: (a) a first soluble fusion protein comprising an: an interleukin-18 (IL-18) polypeptide domain; an IL-15 receptor alpha sushi-binding domain (IL-15RαSu); an immunoglobulin crystallizable fragment (Fc) domain; and an IL-12 domain comprising p40 and p35 subunits; and (b) a second soluble protein comprising IL-15 (IL-15); wherein the IL-15-RαSu domain of the first soluble fusion protein binds to the IL-15 of the second soluble protein to form the soluble fusion protein complex; and wherein the immunoglobulin Fc domain of a first soluble protein complex is covalently linked to the Fc domain of a second soluble protein complex by a disulfide bond. In one aspect, the first soluble fusion protein of the soluble fusion protein complex comprises amino acid sequence set forth in SEQ ID NO:5.

In another aspect, an isolated soluble fusion protein complex is provided comprising a soluble fusion protein, the soluble fusion protein comprising an interleukin-18 (IL-18) polypeptide domain, an immunoglobulin crystallizable fragment (Fc) domain, and an IL-12 polypeptide domain comprising p40 and p35 subunits; wherein the Fc domain of a first soluble protein is covalently linked to the Fc domain of a second soluble protein by a disulfide bond. In one aspect, the soluble fusion protein complex comprises the amino acid sequence set forth in SEQ ID NO:1.

As provided herein, the IL-15 polypeptide domain is an IL-15 variant comprising an N72D mutation (IL-15N72D).

Further as provided herein, the IL-18 polypeptide domain is linked to the IL-15RαSu domain by a flexible linker or is linked by a flexible polypeptide linker to the Ig Fc domain.

Also provided herein, the p40 and p35 subunits of the IL-12 domain are linked by a flexible polypeptide linker. Still further, the IL-12 p40 subunit is linked by a flexible polypeptide linker to the homodimeric Ig Fc domain.

Also provided herein are compositions comprising the soluble fusion protein complexes disclosed herein.

In another aspect, a method of generating a memory-like cytokine enhanced natural killer (M-CENK or M-ceNK) cell is provided. The method comprises o obtaining a plurality of mononuclear cells, and contacting the plurality of mononuclear cells with a corticosteroid; incubating the plurality of mononuclear cells in the presence of the corticosteroid to enrich the mononuclear cells in NK cells; and inducing the enriched NK cells with a soluble fusion protein complex disclosed herein or an IL-15 or derivative thereof and a soluble fusion protein complex disclosed herein to generate the M-CENK cells. In one aspect, the corticosteroid is hydrocortisone.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present or utilized, or combined with other elements, components, or steps that are not expressly referenced. Where the specification or claims refer to at least one of something selected from the group consisting of A, B, C . . . and N, the text should be interpreted as requiring only one element from the group, not A plus N, or B plus N, etc.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

One skilled in the art would understand that descriptions of making and using the particles described herein is for the sole purpose of illustration, and that the present disclosure is not limited by this illustration.

### Example 1: MAXCYTE® Transient Transfection

For transient expression of IL-18/12 SK and IL-18/15/12 SK by MAXCYTE® transfection, CHO-S cells were cultured in suspension in CD-CHO media supplemented with 8 mM L-glutamine in shaker flasks at 37° C. with 125 rpm rotation and 8% CO₂. For transfection, cells in the exponential growth stage were pelleted by centrifugation at 1,400 rpm for 10 min, re-suspended in 10 mL of electroporation buffer, and re-pelleted at 1,400 rpm for 5 min. The cell pellet was resuspended at a density of 2×10⁸ cells/mL in electroporation buffer, mixed with the plasmid harboring either the IL18/12 SK or IL18/15/12 SK sequence at a concentration of 150 μg/mL, and transfected using OC-400 processing assemblies in a MAXCYTE® EXPERT ATx Transfection System. Transfected cells were incubated for 30 min at 37° C., 5% CO₂ and then resuspended in Efficient Feed A Cocktail (CHO-CD EFFICIENTFEED™ A+0.2% Pluronic F-68+1% HT Supplement+1% L-glutamine) at a density of ˜4-6×10⁶ cells/mL. This cell culture was incubated at 37° C. with 5% CO₂ and 125 rpm rotation overnight, 1 mM sodium butyrate was added, and the culture was further incubated at 32° C. with 3% CO₂ and 125 rpm for 13 more days; during this incubation period, MAXCYTE® Feed Cocktail (13.9% CD Hydrolysate, 69.5% CHO CD EFFICIENTFEED™ A, 6.2% Glucose, 6.9% FUNCTIONMAX™ Titer Enhancer, 3.5% L-Glutamine) was added at 10% of the culture volume on Days 4 and 8.

### Purification of IL18/12 SK and IL18/15/12 SK

MAXCYTE® transfection cell culture medium was centrifuged and filtered through a 0.22 μm filter to remove cells and debris, then loaded onto a HITRAP™ MABSELECT SURE™ column (CYTIVA®) on the AKTA Pure system pre-equilibrated with 10 mM Na Phosphate and 150 mM NaCl at pH 7.0. After loading, the column was washed with 10 column volumes of the same buffer. The protein was eluted with 100 mM sodium acetate, pH 3.6, then immediately neutralized using 2 M Tris pH 8.0. The elution fractions were pooled and dialyzed into 10 mM Hepes and 150 mM sodium chloride at pH 7.4.

A production comparison between IL-12/15/18 SK (Altor) (ALTOR BIOSCIENCE®) design vs. IL-18/15/12 SK and IL-18/12 SK designs is shown in Table 1 using the MAXCYTE® transient expression system followed by purification on a Protein A column, as described above. The IL-12/15/18 SK (Altor) (ALTOR BIOSCIENCE®) production yield a very low titer of about 50 μg/ml (0.047 g/L at harvest) and only a 1.9% recovery (see bolded amounts in the “IL-12/15/18 SK (Altor)” column of Table 1).

TABLE 1
Production Comparison

| | IL-12/15/18 | | IL-18/15/12 | | IL-18/12 | |
|---|---|---|---|---|---|---|
| | SK (Altor) | | SK | | SK | |
| Titer (g/L) at harvest | 0.047 | | 0.097 | | 0.289 | |
| Culture volume at harvest | 80 | mL | 110 | mL | 45 | mL |
| Expected mass | 3.8 | mg | 10.7 | mg | 13.0 | mg |
| Mass recovered | 0.073 | mg | 3.4 | mg | 4.0 | mg |
| % recovery | 1.9% | | 31.7% | | 30% | |
| SEC-HPLC | 43.6/56.4/0 | | 38.7/58.1/3.2 | | 43.8/56.2 | |
| % HWM/% Main/% LMW | | | | | | |

### Example 2-Generation of M-CENK Cells

Fresh or cryopreserved/thawed apheresis product (patient-derived mononuclear cells) are expanded in NK-GM media (NK-MACS® Basal Medium with NK-MACS® Supplement and 10% human AB serum) supplemented with N-803 (0.8 nM) and Hydrocortisone (1 μM). Once the culture reaches at least 85% CD56 positive cells up to expansion day 20 at a density of at least 1×10⁶ cells/mL, fixed concentrations of 1) N-803 (100-300 ng/ml), IL-12 (1-100 ng/ml) and IL-18 (5-250 ng/mL), or 2)N-803 (100-300 ng/ml) and IL12/18 SK (5 μg/ml) are added to the culture. Cells are stimulated with the above cytokines for 14-16 hours which induces the memory like phenotype of the CD56 positive cells. After completion of the M-CENK induction stage, cells are washed using Sepax C-Pro device. Most typically, a 5% albumin (human) solution is used as washing and resuspension solution. For cryopreservation, M-CENK cells are formulated in media containing 5% Albumin (human) USP: CryoStor 10 (CS10) (1:1). M-CENK have enhanced ability to kill cancer cell targets through their increased IFN-γ production. In addition, these cells are phenotypically CD56+, CD25+, DNAM-1+, and NKP30+, NKG2D+, NKG2A+, and CD3−.

### Example 3-Stimulation of CENK with Cytokine Cocktail (Human IL-12, IL-18 and N-803)

The NANT001 Bioreactor platform system (IMMUNITYBIO®, INC.) is a self-contained bioreactor that executes pre-programmed protocols that instruct automatic procedures and real time monitoring throughout the entirety of the recovery, enrichment, expansion and M-CENK induction stages of the manufacturing process. Programmable process parameters include pH monitoring, cell imaging, temperature, and rocking parameters. The NANT001 Bioreactor includes a thermostatic compartment, a touch-screen user interface, a barcode reader, a pH Estimation unit, an integrated imaging system, and a gas flow control. Components are easy to load, single-use closed-system design for safe and cGMP-compliant cell processing and include a waste bag, up to 4 L, an aseptic disconnector, a harvesting bottle, auxiliary bags×2, up to 100 mL ea., aseptic connectors, a cell culture flask, 636 cm², a media bag, up to 3 L, and a buffer bag, up to 3 L. Exemplary systems suitable for use herein are described, for example, in U.S. Pat. No. 10,801,005 and US 2017/0037357, incorporated by reference herein.

Once the NANT001 bioreactor culture of expanded apheresis product reaches ≥85% CD56 positive cells up to expansion day 20 at a density of ≥1×10⁶ cells/mL, fixed concentrations of 1) N-803 (100-300 ng/mL), IL-12 (1-100 ng/ml) and IL-18 (5-250 ng/mL) or 2) N-803 (100-300 ng/ml) and IL12/18 superkine (5 μg/ml) are added to the culture in fresh NK-Growth Medium (NK-GM) up to a final total culture volume of approximately 650 mL. Cells are stimulated with the cytokines for 14-16 hours which induces a memory like phenotype of the CD56 positive cells (M-CENK) contained within the NANT001 Bioreactor. This stimulation with cytokines in the bioreactor is then terminated by bulk cell harvesting of the culture using the NANT 001 auto-export feature. Samples are taken for mycoplasma testing prior to culture harvest.

### Sequences:

IL-18/12 superkine protein complex
SEQ ID NO: 1 (IL-18/12 superkine; amino acid)-leader peptide not included (see SEQ
ID NO: 8)
YFGKLESKLSVIRNLNDQVLFIDQGNRPLFEDMTDSDCRDNAPRTIFIISMYKDSQP
RGMAVTISVKCEKISTLSCENKIISFKEMNPPDNIKDTKSDIIFFQRSVPGHDNKMQ
FESSSYEGYFLACEKERDLFKLILKKEDELGDRSIMFTVQNEDGGGGSGGGGSGG
GGSGGGGSDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEAL
HNHYTQKSLSLSPGKGGGGSGGGGSGGGGSGGGGSIWELKKDVYVVELDWYPD
APGEMVVLTCDTPEEDGITWTLDQSSEVLGSGKTLTIQVKEFGDAGQYTCHKGGE
VLSHSLLLLHKKEDGIWSTDILKDQKEPKNKTFLRCEAKNYSGRFTCWWLTTIST
DLTFSVKSSRGSSDPQGVTCGAATLSAERVRGDNKEYEYSVECQEDSACPAAEES
LPIEVMVDAVHKLKYENYTSSFFIRDIIKPDPPKNLQLKPLKNSRQVEVSWEYPDT
WSTPHSYFSLTFCVQVQGKSKREKKDRVFTDKTSATVICRKNASISVRAQDRYYS
SSWSEWASVPCSGGGGSGGGGSGGGGSRNLPVATPDPGMFPCLHHSQNLLRAVS
NMLQKARQTLEFYPCTSEEIDHEDITKDKTSTVEACLPLELTKNESCLNSRETSFIT
NGSCLASRKTSFMMALCLSSIYEDLKMYQVEFKTMNAKLLMDPKRQIFLDQNML
AVIDELMQALNFNSETVPQKSSLEEPDFYKTKIKLCILLHAFRIRAVTIDRVMSYLN
AS
SEQ ID NO: 2 (IL-18/12 superkine; nucleic acid)-leader sequence not included (see
SEQ ID NO: 9)
tacttcggcaagctggagtccaagctgtccgtgatcaggaacctgaacgaccaggtgctgttcatcgaccagggcaacaggccc
ctgttcgaggacatgaccgactccgactgcagggacaacgcccctaggaccatcttcatcatctccatgtataaggacagccagc
ccaggggaatggccgtgaccatctccgtgaagtgcgagaagatctccaccctgtcctgcgagaacaagatcatctccttcaagga
gatgaacccccccgacaacatcaaggacaccaagtccgacatcatcttcttccagcggtccgtgcccggacacgacaacaagat
gcagttcgagtcctcctcctacgagggctactttctggcctgtgagaaggagagggacctcttcaagctcatcctgaagaaggag
gacgagctgggcgacaggtccatcatgttcaccgtgcagaacgaggacggtggaggtggctccggTggcggAggaagtggt
ggAggcggATCTggTggAggAggcagcgataaaacacatacttgcccaccttgtcctgcaccagagctgctgggaggt
ccaagcgtgttcctgtttccacccaagcccaaagacacactgatgatttctcgaactccagaagtgacctgcgtggtcgtggacgt
gagccacgaggatcccgaagtgaagttcaactggtacgtggatggcgtggaggtccataatgctaagactaaaccccgggagg
aacagtacaacagcacttatagggtcgtgtccgtgctgaccgtcctgcatcaggattggctgaacgggaaggaatataagtgcaa
agtgtctaataaggcactgcccgcccctatcgagaaaacaattagcaaggctaagggtcagcctagagaaccacaggtgtacact
ctgcctccaagtcgcgacgagctgaccaagaaccaggtgtcactgacatgtctggtcaaaggcttttatccctcagatatcgcagt
ggagtgggaaagcaatggacagcctgagaacaattacaagaccacaccccctgtgctggacagtgatggctcattctttctgtatt
ctaagctgacagtggacaaaagtaggtggcagcagggaaacgtgttctcctgctctgtcatgcacgaagcactgcacaatcattat
acccagaagagtctgtcactgagccctggcaaaggaggtggTggatcaggTggAggAggaagtggTggaggAggAtc
TggAggTggTggAAGTatctgggagctgaagaaagacgtgtatgtcgtggagctggactggtatcctgacgcccccggc
gagatggtggtgctgacatgcgacacccctgaggaggatggcatcacatggaccctggaccaaagcagcgaggtgctgggctc
cggaaagaccctgaccatccaggtgaaggagttcggcgacgccggccagtatacctgccataagggaggcgaggtgctgtcc
cactccctgctcctgctgcacaagaaggaagatggcatctggagcaccgatattctgaaggaccagaaggagcccaagaacaa
aacctttctgcggtgcgaggccaagaattattccggcaggttcacctgctggtggctgaccacaatctccaccgacctgaccttca
gcgtcaagagctccaggggatcctccgatcctcagggcgtgacctgtggagctgccaccctgtccgctgagagggtgaggggc
gacaacaaggagtacgagtactccgtcgagtgtcaggaggactccgcctgccctgctgccgaagagagcctgcctatcgaagtc
atggtggacgccgtgcacaagctgaagtatgagaactacaccagcagcttcttcatccgggacattatcaagcctgatccccctaa
gaacctgcagctcaagcccctgaagaattcccggcaagtcgaggtgtcctgggagtaccccgacacctggtccacccctcactc
ctattttagcctgaccttctgcgtgcaggtgcagggcaagagcaagagggagaagaaagaccgggtgttcaccgacaagacca
gcgctaccgtgatctgtcggaagaacgcttccatttccgtgcgggctcaggacaggtattactcctcctcctggtccgagtgggcta
gcgtcccctgcagcggaggtggcggatccggaggtggaggttctggtggaggtgggagtaggaacctgcccgtggctacacc
cgaccctggaatgttcccctgtctccaccacagccaaaacctcctgcgggccgtgtccaacatgctgcaaaaggctcggcagac
actggagttctacccctgcaccagcgaggagatcgaccatgaggacatcacaaaggacaagacaagcaccgtggaggcttgcc
tccccctggaactgaccaagaatgagtcctgcctcaacagccgggagacatccttcatcaccaatggctcctgtctggcttcccgg
aagacaagcttcatgatggccctgtgcctgtccagcatctatgaggacctgaagatgtaccaggtcgagtttaagaccatgaacgc
caagctgctgatggaccccaagcggcaaatcttcctggaccagaacatgctggctgtgatcgacgagctgatgcaggctctgaa
cttcaacagcgagaccgtgccccagaagtcctccctggaggagcctgatttttacaagaccaaaatcaagctctgcatcctcctgc
acgccttccggatcagggccgtgaccatcgatcgggtgatgtcctacctgaatgcttcctga
IL-18/15/12 superkine
SEQ ID NO: 3 (IL15N72D; amino acid) leader peptide not included (see SEQ ID NO: 8)
NWVNVISDLKKIEDLIQSMHIDATLYTESDVHPSCKVTAMKCFLLELQVISLESGD
ASIHDTVENLIILANDSLSSNGNVTESGCKECEELEEKNIKEFLQSFVHIVQMFINTS
SEQ ID NO: 4 (IL15N72D; nucleic acid)
Aactgggtgaatgtaataagtgatttgaaaaaaattgaagatcttatccagtccatgcacatcgacgccaccctgtacaccgagag
cgacgtgcacccctcctgcaaggtgaccgccatgaagtgcttcctgctggagctgcaggtgatctccctggagtccggcgacgc
ctccatccacgacaccgtggagaacctgatcatcctggccaacgactccctgtcctccaacggcaacgtgaccgagtccggctg
caaggagtgcgaggagctggaggagaagaacatcaaggagttcctgcagtccttcgtgcacatcgtccaaatgttcatcaacact
tct
SEQ ID NO: 5 (IL18/IL15RaSushi/IL12; amino acid)
YFGKLESKLSVIRNLNDQVLFIDQGNRPLFEDMTDSDCRDNAPRTIFIISMYKDSQP
RGMAVTISVKCEKISTLSCENKIISFKEMNPPDNIKDTKSDIIFFQRSVPGHDNKMQ
FESSSYEGYFLACEKERDLFKLILKKEDELGDRSIMFTVQNEDGGGGSGGGGSGG
GGSGGGGSGGGGSGGGGSITCPPPMSVEHADIWVKSYSLYSRERYICNSGFKRKA
GTSSLTECVLNKATNVAHWTTPSLKCIREPKSSDKTHTCPPCPAPELLGGPSVFLFP
PKPKDTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSK
LTVDKSRWQQGNVFSCSVMHEALHNHYTQKSLSLSPGKGGGGSGGGGSGGGGS
GGGGSIWELKKDVYVVELDWYPDAPGEMVVLTCDTPEEDGITWTLDQSSEVLGS
GKTLTIQVKEFGDAGQYTCHKGGEVLSHSLLLLHKKEDGIWSTDILKDQKEPKNK
TFLRCEAKNYSGRFTCWWLTTISTDLTFSVKSSRGSSDPQGVTCGAATLSAERVR
GDNKEYEYSVECQEDSACPAAEESLPIEVMVDAVHKLKYENYTSSFFIRDIIKPDPP
KNLQLKPLKNSRQVEVSWEYPDTWSTPHSYFSLTFCVQVQGKSKREKKDRVFTD
KTSATVICRKNASISVRAQDRYYSSSWSEWASVPCSGGGGSGGGGSGGGGSRNLP
VATPDPGMFPCLHHSQNLLRAVSNMLQKARQTLEFYPCTSEEIDHEDITKDKTST
VEACLPLELTKNESCLNSRETSFITNGSCLASRKTSFMMALCLSSIYEDLKMYQVE
FKTMNAKLLMDPKRQIFLDQNMLAVIDELMQALNFNSETVPQKSSLEEPDFYKTK
IKLCILLHAFRIRAVTIDRVMSYLNAS
SEQ ID NO: 6 (IL18/IL15RaSushi/IL12; nucleic acid)
tacttcggcaagctggagtccaagctgtccgtgatcaggaacctgaacgaccaggtgctgttcatcgaccagggcaacaggccc
ctgttcgaggacatgaccgactccgactgcagggacaacgcccctaggaccatcttcatcatctccatgtataaggacagccagc
ccaggggaatggccgtgaccatctccgtgaagtgcgagaagatctccaccctgtcctgcgagaacaagatcatctccttcaagga
gatgaacccccccgacaacatcaaggacaccaagtccgacatcatcttcttccagcggtccgtgcccggacacgacaacaagat
gcagttcgagtcctcctcctacgagggctactttctggcctgtgagaaggagagggacctcttcaagctcatcctgaagaaggag
gacgagctgggcgacaggtccatcatgttcaccgtgcagaacgaggacggaggtggtggaagtggaggtggaggatctggtg
gtggcggatcaggtggcggaggatccggtggaggaggttctggaggaggaggtagcatcacgtgtcctcctcctatgtccgtgg
aacacgcagacatctgggtcaagagctacagcttgtactccagggagcggtacatttgtaactctggtttcaagcgtaaagccggc
acgtccagcctgacggagtgcgtgttgaacaaggccacgaatgtcgcccactggacaacccccagtctcaaatgcattagagaa
ccaaagtcctccgataaaacacatacttgcccaccttgtcctgcaccagagctgctgggaggtccaagcgtgttcctgtttccaccc
aagcccaaagacacactgatgatttctcgaactccagaagtgacctgcgtggtcgtggacgtgagccacgaggatcccgaagtg
aagttcaactggtacgtggatggcgtggaggtccataatgctaagactaaaccccgggaggaacagtacaacagcacttatagg
gtcgtgtccgtgctgaccgtcctgcatcaggattggctgaacgggaaggaatataagtgcaaagtgtctaataaggcactgcccg
cccctatcgagaaaacaattagcaaggctaagggtcagcctagagaaccacaggtgtacactctgcctccaagtcgcgacgagc
tgaccaagaaccaggtgtcactgacatgtctggtcaaaggcttttatccctcagatatcgcagtggagtgggaaagcaatggacag
cctgagaacaattacaagaccacaccccctgtgctggacagtgatggctcattctttctgtattctaagctgacagtggacaaaagt
aggtggcagcagggaaacgtgttctcctgctctgtcatgcacgaagcactgcacaatcattatacccagaagagtctgtcactgag
ccctggcaaaggaggtggtggatcaggtggaggaggaagtggtggaggaggatctggaggtggtggaagtatctgggagctg
aagaaagacgtgtatgtcgtggagctggactggtatcctgacgcccccggcgagatggtggtgctgacatgcgacacccctgag
gaggatggcatcacatggaccctggaccaaagcagcgaggtgctgggctccggaaagaccctgaccatccaggtgaaggagt
tcggcgacgccggccagtatacctgccataagggaggcgaggtgctgtcccactccctgctcctgctgcacaagaaggaagat
ggcatctggagcaccgatattctgaaggaccagaaggagcccaagaacaaaacctttctgcggtgcgaggccaagaattattcc
ggcaggttcacctgctggtggctgaccacaatctccaccgacctgaccttcagcgtcaagagctccaggggatcctccgatcctc
agggcgtgacctgtggagctgccaccctgtccgctgagagggtgaggggcgacaacaaggagtacgagtactccgtcgagtgt
caggaggactccgcctgccctgctgccgaagagagcctgcctatcgaagtcatggtggacgccgtgcacaagctgaagtatga
gaactacaccagcagcttcttcatccgggacattatcaagcctgatccccctaagaacctgcagctcaagcccctgaagaattccc
ggcaagtcgaggtgtcctgggagtaccccgacacctggtccacccctcactcctattttagcctgaccttctgcgtgcaggtgcag
ggcaagagcaagagggagaagaaagaccgggtgttcaccgacaagaccagcgctaccgtgatctgtcggaagaacgcttcca
tttccgtgcgggctcaggacaggtattactcctcctcctggtccgagtgggctagcgtcccctgcagcggaggtggcggatccgg
aggtggaggttctggtggaggtgggagtaggaacctgcccgtggctacacccgaccctggaatgttcccctgtctccaccacag
ccaaaacctcctgcgggccgtgtccaacatgctgcaaaaggctcggcagacactggagttctacccctgcaccagcgaggagat
cgaccatgaggacatcacaaaggacaagacaagcaccgtggaggcttgcctccccctggaactgaccaagaatgagtcctgcc
tcaacagccgggagacatccttcatcaccaatggctcctgtctggcttcccggaagacaagcttcatgatggccctgtgcctgtcc
agcatctatgaggacctgaagatgtaccaggtcgagtttaagaccatgaacgccaagctgctgatggaccccaagcggcaaatct
tcctggaccagaacatgctggctgtgatcgacgagctgatgcaggctctgaacttcaacagcgagaccgtgccccagaagtcctc
cctggaggagcctgatttttacaagaccaaaatcaagctctgcatcctcctgcacgccttccggatcagggccgtgaccatcgatc
gggtgatgtcctacctgaatgcttcctga
SEQ ID NO: 7 IL-15Rα sushi domain amino acid sequence
ITCPPPMSVEHADIWVKSYSLYSRERYICNSGFKRKAGTSSLTECVLNKATNVAH
WTTPSLKCIR
SEQ ID NO: 8 (IL-18/12 superkine and IL15N72D leader peptide sequence; amino acid)
MEWSWVFLFFLSVTTGVHS
SEQ ID NO: 9 (IL-18/12 superkine leader sequence; nucleic acid)
atggaatggagctgggtctttctcttcttcctgtcagtaaccaccggtgtccactcc

What is claimed is:

1. An isolated soluble-fusion protein complex comprising two soluble proteins,
wherein the complex comprises:
a. a first soluble fusion protein comprising an:
an interleukin-18 (IL-18) polypeptide domain;
an IL-15 receptor alpha sushi-binding domain (IL-15RαSu);
an immunoglobulin (Ig) crystallizable fragment (Fc) domain; and
an IL-12 polypeptide domain comprising p40 and p35 subunits; and
b. a second soluble protein comprising IL-15 (IL-15);
wherein the IL-15-RαSu domain of the first soluble fusion protein binds to the IL-15 of the second soluble protein to form the soluble fusion protein complex; and
wherein the immunoglobulin Fc domain of a first soluble protein complex is covalently linked to the Fc domain of a second soluble protein complex by a disulfide bond.

2. The soluble fusion protein complex of claim 1, wherein the IL-15 polypeptide domain is an IL-15 variant comprising an asparagine-to-aspartate mutation at amino acid position 72 (IL-15N72D).

3. The soluble fusion protein complex of claim 1, wherein the IL-18 polypeptide domain is linked to the IL-15RαSu domain by a flexible polypeptide linker.

4. The soluble fusion protein complex of claim 1, wherein the p40 and p35 subunits of the IL-12 domain are linked by a flexible polypeptide linker.

5. The soluble fusion protein complex of claim 1, wherein the IL-12 p40 subunit is linked by a flexible polypeptide linker to the Ig Fc domain.

6. The soluble fusion protein complex of claim 1, wherein the first soluble fusion protein comprises the amino acid sequence set forth in SEQ ID NO:5.

7. An isolated soluble fusion protein complex comprising a soluble fusion protein, the soluble fusion protein comprising an interleukin-18 (IL-18) polypeptide domain, an Ig crystallizable fragment (Fc) domain, and an IL-12 polypeptide domain comprising p40 and p35 subunits; wherein the Fc domain of a first soluble protein is covalently linked to the Fc domain of a second soluble protein by a disulfide bond.

8. The soluble fusion protein complex of claim 7, wherein the IL-15 polypeptide domain is an IL-15 variant comprising an N72D mutation (IL-15N72D).

9. The soluble fusion protein complex of claim 7, wherein the IL-18 polypeptide domain is linked by a flexible polypeptide linker to the Ig Fc domain.

10. The soluble fusion protein complex of claim 7, wherein the IL-12 p40 subunit is linked by a flexible polypeptide linker to the Ig Fc domain.

11. The soluble fusion protein complex of claim 7, wherein the soluble fusion protein complex comprises the amino acid sequence set forth in SEQ ID NO:1.

12. A method of generating a memory-like cytokine enhanced natural killer (M-CENK) cell, comprising:
a. obtaining a plurality of mononuclear cells, and contacting the plurality of mononuclear cells with a corticosteroid;
b. incubating the plurality of mononuclear cells in the presence of the corticosteroid to enrich the mononuclear cells in NK cells; and
c. inducing the enriched NK cells with i) the soluble fusion protein complex of claim 1 or ii) IL-15 or a derivative thereof and the soluble fusion protein complex of claim 7 to generate the M-CENK cells.

13. The method of claim 12 wherein the corticosteroid is hydrocortisone.

14. The method of claim 12, wherein the IL-15 derivative is N-803.


SEQUENCE LISTING (9 sequences)

SEQ ID NO: 1 - protein, 942 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDGGGGSGGGGSGGGGSGGGGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 2 - DNA, 2829 bp, synthetic construct
  source 1..2829: mol_type = other DNA
tacttcggcaagctggagtccaagctgtccgtgatcaggaacctgaacgaccaggtgctgttcatcgaccagggcaacaggcccctgttcgaggacatgaccgactccgactgcagggacaacgcccctaggaccatcttcatcatctccatgtataaggacagccagcccaggggaatggccgtgaccatctccgtgaagtgcgagaagatctccaccctgtcctgcgagaacaagatcatctccttcaaggagatgaacccccccgacaacatcaaggacaccaagtccgacatcatcttcttccagcggtccgtgcccggacacgacaacaagatgcagttcgagtcctcctcctacgagggctactttctggcctgtgagaaggagagggacctcttcaagctcatcctgaagaaggaggacgagctgggcgacaggtccatcatgttcaccgtgcagaacgaggacggtggaggtggctccggtggcggaggaagtggtggaggcggatctggtggaggaggcagcgataaaacacatacttgcccaccttgtcctgcaccagagctgctgggaggtccaagcgtgttcctgtttccacccaagcccaaagacacactgatgatttctcgaactccagaagtgacctgcgtggtcgtggacgtgagccacgaggatcccgaagtgaagttcaactggtacgtggatggcgtggaggtccataatgctaagactaaaccccgggaggaacagtacaacagcacttatagggtcgtgtccgtgctgaccgtcctgcatcaggattggctgaacgggaaggaatataagtgcaaagtgtctaataaggcactgcccgcccctatcgagaaaacaattagcaaggctaagggtcagcctagagaaccacaggtgtacactctgcctccaagtcgcgacgagctgaccaagaaccaggtgtcactgacatgtctggtcaaaggcttttatccctcagatatcgcagtggagtgggaaagcaatggacagcctgagaacaattacaagaccacaccccctgtgctggacagtgatggctcattctttctgtattctaagctgacagtggacaaaagtaggtggcagcagggaaacgtgttctcctgctctgtcatgcacgaagcactgcacaatcattatacccagaagagtctgtcactgagccctggcaaaggaggtggtggatcaggtggaggaggaagtggtggaggaggatctggaggtggtggaagtatctgggagctgaagaaagacgtgtatgtcgtggagctggactggtatcctgacgcccccggcgagatggtggtgctgacatgcgacacccctgaggaggatggcatcacatggaccctggaccaaagcagcgaggtgctgggctccggaaagaccctgaccatccaggtgaaggagttcggcgacgccggccagtatacctgccataagggaggcgaggtgctgtcccactccctgctcctgctgcacaagaaggaagatggcatctggagcaccgatattctgaaggaccagaaggagcccaagaacaaaacctttctgcggtgcgaggccaagaattattccggcaggttcacctgctggtggctgaccacaatctccaccgacctgaccttcagcgtcaagagctccaggggatcctccgatcctcagggcgtgacctgtggagctgccaccctgtccgctgagagggtgaggggcgacaacaaggagtacgagtactccgtcgagtgtcaggaggactccgcctgccctgctgccgaagagagcctgcctatcgaagtcatggtggacgccgtgcacaagctgaagtatgagaactacaccagcagcttcttcatccgggacattatcaagcctgatccccctaagaacctgcagctcaagcccctgaagaattcccggcaagtcgaggtgtcctgggagtaccccgacacctggtccacccctcactcctattttagcctgaccttctgcgtgcaggtgcagggcaagagcaagagggagaagaaagaccgggtgttcaccgacaagaccagcgctaccgtgatctgtcggaagaacgcttccatttccgtgcgggctcaggacaggtattactcctcctcctggtccgagtgggctagcgtcccctgcagcggaggtggcggatccggaggtggaggttctggtggaggtgggagtaggaacctgcccgtggctacacccgaccctggaatgttcccctgtctccaccacagccaaaacctcctgcgggccgtgtccaacatgctgcaaaaggctcggcagacactggagttctacccctgcaccagcgaggagatcgaccatgaggacatcacaaaggacaagacaagcaccgtggaggcttgcctccccctggaactgaccaagaatgagtcctgcctcaacagccgggagacatccttcatcaccaatggctcctgtctggcttcccggaagacaagcttcatgatggccctgtgcctgtccagcatctatgaggacctgaagatgtaccaggtcgagtttaagaccatgaacgccaagctgctgatggaccccaagcggcaaatcttcctggaccagaacatgctggctgtgatcgacgagctgatgcaggctctgaacttcaacagcgagaccgtgccccagaagtcctccctggaggagcctgatttttacaagaccaaaatcaagctctgcatcctcctgcacgccttccggatcagggccgtgaccatcgatcgggtgatgtcctacctgaatgcttcctga

SEQ ID NO: 3 - protein, 114 aa, synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 4 - DNA, 342 bp, synthetic construct
  source 1..342: mol_type = other DNA
aactgggtgaatgtaataagtgatttgaaaaaaattgaagatcttatccagtccatgcacatcgacgccaccctgtacaccgagagcgacgtgcacccctcctgcaaggtgaccgccatgaagtgcttcctgctggagctgcaggtgatctccctggagtccggcgacgcctccatccacgacaccgtggagaacctgatcatcctggccaacgactccctgtcctccaacggcaacgtgaccgagtccggctgcaaggagtgcgaggagctggaggagaagaacatcaaggagttcctgcagtccttcgtgcacatcgtccaaatgttcatcaacacttct

SEQ ID NO: 5 - protein, 1022 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDGGGGSGGGGSGGGGSGGGGSGGGGSGGGGSITCPPPMSVEHADIWVKSYSLYSRERYICNSGFKRKAGTSSLTECVLNKATNVAHWTTPSLKCIREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSGGGGSGGGGSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 6 - DNA, 3069 bp, synthetic construct
  source 1..3069: mol_type = other DNA
tacttcggcaagctggagtccaagctgtccgtgatcaggaacctgaacgaccaggtgctgttcatcgaccagggcaacaggcccctgttcgaggacatgaccgactccgactgcagggacaacgcccctaggaccatcttcatcatctccatgtataaggacagccagcccaggggaatggccgtgaccatctccgtgaagtgcgagaagatctccaccctgtcctgcgagaacaagatcatctccttcaaggagatgaacccccccgacaacatcaaggacaccaagtccgacatcatcttcttccagcggtccgtgcccggacacgacaacaagatgcagttcgagtcctcctcctacgagggctactttctggcctgtgagaaggagagggacctcttcaagctcatcctgaagaaggaggacgagctgggcgacaggtccatcatgttcaccgtgcagaacgaggacggaggtggtggaagtggaggtggaggatctggtggtggcggatcaggtggcggaggatccggtggaggaggttctggaggaggaggtagcatcacgtgtcctcctcctatgtccgtggaacacgcagacatctgggtcaagagctacagcttgtactccagggagcggtacatttgtaactctggtttcaagcgtaaagccggcacgtccagcctgacggagtgcgtgttgaacaaggccacgaatgtcgcccactggacaacccccagtctcaaatgcattagagaaccaaagtcctccgataaaacacatacttgcccaccttgtcctgcaccagagctgctgggaggtccaagcgtgttcctgtttccacccaagcccaaagacacactgatgatttctcgaactccagaagtgacctgcgtggtcgtggacgtgagccacgaggatcccgaagtgaagttcaactggtacgtggatggcgtggaggtccataatgctaagactaaaccccgggaggaacagtacaacagcacttatagggtcgtgtccgtgctgaccgtcctgcatcaggattggctgaacgggaaggaatataagtgcaaagtgtctaataaggcactgcccgcccctatcgagaaaacaattagcaaggctaagggtcagcctagagaaccacaggtgtacactctgcctccaagtcgcgacgagctgaccaagaaccaggtgtcactgacatgtctggtcaaaggcttttatccctcagatatcgcagtggagtgggaaagcaatggacagcctgagaacaattacaagaccacaccccctgtgctggacagtgatggctcattctttctgtattctaagctgacagtggacaaaagtaggtggcagcagggaaacgtgttctcctgctctgtcatgcacgaagcactgcacaatcattatacccagaagagtctgtcactgagccctggcaaaggaggtggtggatcaggtggaggaggaagtggtggaggaggatctggaggtggtggaagtatctgggagctgaagaaagacgtgtatgtcgtggagctggactggtatcctgacgcccccggcgagatggtggtgctgacatgcgacacccctgaggaggatggcatcacatggaccctggaccaaagcagcgaggtgctgggctccggaaagaccctgaccatccaggtgaaggagttcggcgacgccggccagtatacctgccataagggaggcgaggtgctgtcccactccctgctcctgctgcacaagaaggaagatggcatctggagcaccgatattctgaaggaccagaaggagcccaagaacaaaacctttctgcggtgcgaggccaagaattattccggcaggttcacctgctggtggctgaccacaatctccaccgacctgaccttcagcgtcaagagctccaggggatcctccgatcctcagggcgtgacctgtggagctgccaccctgtccgctgagagggtgaggggcgacaacaaggagtacgagtactccgtcgagtgtcaggaggactccgcctgccctgctgccgaagagagcctgcctatcgaagtcatggtggacgccgtgcacaagctgaagtatgagaactacaccagcagcttcttcatccgggacattatcaagcctgatccccctaagaacctgcagctcaagcccctgaagaattcccggcaagtcgaggtgtcctgggagtaccccgacacctggtccacccctcactcctattttagcctgaccttctgcgtgcaggtgcagggcaagagcaagagggagaagaaagaccgggtgttcaccgacaagaccagcgctaccgtgatctgtcggaagaacgcttccatttccgtgcgggctcaggacaggtattactcctcctcctggtccgagtgggctagcgtcccctgcagcggaggtggcggatccggaggtggaggttctggtggaggtgggagtaggaacctgcccgtggctacacccgaccctggaatgttcccctgtctccaccacagccaaaacctcctgcgggccgtgtccaacatgctgcaaaaggctcggcagacactggagttctacccctgcaccagcgaggagatcgaccatgaggacatcacaaaggacaagacaagcaccgtggaggcttgcctccccctggaactgaccaagaatgagtcctgcctcaacagccgggagacatccttcatcaccaatggctcctgtctggcttcccggaagacaagcttcatgatggccctgtgcctgtccagcatctatgaggacctgaagatgtaccaggtcgagtttaagaccatgaacgccaagctgctgatggaccccaagcggcaaatcttcctggaccagaacatgctggctgtgatcgacgagctgatgcaggctctgaacttcaacagcgagaccgtgccccagaagtcctccctggaggagcctgatttttacaagaccaaaatcaagctctgcatcctcctgcacgccttccggatcagggccgtgaccatcgatcgggtgatgtcctacctgaatgcttcctga

SEQ ID NO: 7 - protein, 65 aa, synthetic construct
ITCPPPMSVEHADIWVKSYSLYSRERYICNSGFKRKAGTSSLTECVLNKATNVAHWTTPSLKCIR

SEQ ID NO: 8 - protein, 19 aa, synthetic construct
MEWSWVFLFFLSVTTGVHS

SEQ ID NO: 9 - DNA, 57 bp, synthetic construct
  source 1..57: mol_type = other DNA
atggaatggagctgggtctttctcttcttcctgtcagtaaccaccggtgtccactcc